(19) United States
(12) Patent Application Publication
(10) Pub. No.: US 2021/0244769 A1
(43) Pub. Date: Aug. 12, 2021

(54) PAIN-REDUCING EFFECTS OF FIBROBLASTS AND TREATMENT OF PAIN

(71) Applicant: Figene, LLC, Houston, TX (US)

(72) Inventors: Pete O'Heeron, Houston, TX (US); Thomas Ichim, San Diego, CA (US)

(21) Appl. No.: 17/052,859

(22) PCT Filed: May 3, 2019
(86) PCT No.: PCT/US19/30596

§ 371 Date: Nov. 4, 2020

Related U.S. Application Data
(60) Provisional application No. 62/666,828, filed on May 4, 2018.

(51) Int. Cl. A61K 35/33; C12N 5/077

(52) U.S. Cl. CPC A61K 35/33; C12N 2500/02; C12N 5/0656

(57) ABSTRACT

Embodiments of the disclosure include methods and compositions for treatment or prevention of pain in an individual. In specific embodiments, the methods and compositions encompass fibroblasts and/or fibroblast derivatives for the treatment of any kind of pain, including back pain. In particular aspects, exosomes and/or lysate and/or conditioned media from the fibroblasts are provided to an individual in need thereof.

This application claims priority to U.S. Provisional Patent Application Ser. No. 62/666,828, which is incorporated by reference herein in its entirety.

### TECHNICAL FIELD

Embodiments of the disclosure regard at least the fields of cell biology, molecular biology, biochemistry, and medicine.

### BACKGROUND

Lower back pain (LBP), causes significant impact personally and socially, affecting 70 to 80% of adults least once in a lifetime [1]. In 1998, about 26.3 billion dollars were expended due to LBP in United States [2]. Back pain (BP) is generally self-limited and positive and patients undergoing acute BP may usually become better in one month or can return to work [3, 4]. However, 2 to 7% of the patients show the progress into chronic BP and chronic or regenerative BP may cause 75 to 85% of absence from works [5-7]. Accordingly, in case of occurring acute BP, it is important to apply a method of treating while minimizing side effects to thus relieve pains, which in turn improves functionality, reduces a rate of absence and suppress the progress into chronic BP. Treatment of LBP generally includes prescription of an analgesic agent, for example, acetaminophen, or non-steroid anti-inflammatory drugs (NSAIDs) and also encouraging a patient to continuously retain daily activity [8, 9]. NSAIDs are effective for curing BP a short period of time and, in an aspect of relieving pains, more superior over acetaminophen [10]. In most general, intramuscular application of diclofenac is a method of curing acute pains however, using NSAID often causes side effects in the stomach (and intestines) [11]. There is currently an increasing concern about safety of cyclooxygenase-2 selective NSAIDs for cardiovascular diseases, in particular, thrombotic diseases such as acute myocardial infarction, unstable angina pectoris, cardiac arrest, sudden (cardiac) death [12]. The disclosure provides a means to overcome pain, particularly in individuals with LBP through the intradiscal administration of fibroblasts or derivatives of fibroblasts.

### BRIEF SUMMARY

The present invention is directed to methods and compositions related to the treatment or prevention of pain. In particular embodiments, any type of pain is treated or prevented upon administration of fibroblasts and/or fibroblast derivatives such as extracts of the fibroblasts, lysates of the fibroblasts, and/or nucleic acid compositions thereof.

In particular embodiments the individual has been determined to have a need for the treatment of pain, and in specific embodiments a medical practitioner provides the fibroblasts and/or fibroblast derivatives for the treatment of pain specifically. The delivered fibroblasts and/or fibroblast derivatives may or may not have another therapeutic or preventative aspect in vivo.

The disclosure pertains to the field of pain management, more specifically, the disclosure pertains to the field of utilizing fibroblasts and/or fibroblast derivatives to ameliorate pain by means of administering the fibroblasts and/or fibroblast derivatives into an individual in need of therapy. More specifically, the disclosure encompasses the use of intradiscally-administered fibroblasts to reduce pain, in particular discogenic pain.

In one embodiment, there is a method of treating or preventing pain in an individual, comprising the step of administering to the individual an effective amount of fibroblasts and/or derivatives thereof and/or conditioned media from culture of the fibroblasts. The administration may be local or systemic to the individual. The administration may be to the spine of the individual, and the administration may be intradiscally in the individual. In some cases, the pain is acute or chronic. The individual may be receiving an additional treatment, such as for pain. The pain may be of any kind, including at least a) neuropathic pain; b) nociceptive pain; c) phantom pain; d) psychogenic pain; e) incident pain; f) breakthrough pain; g) discogenic pain; h) idiopathic pain; or i) a combination thereof.

In some embodiments, the fibroblast derivative comprises lysate and/or exosomes, and the exosomes may be obtained following culture of the fibroblasts under suitable conditions.

The fibroblasts may express CXCR-4; CD-271; FGF-1 receptor; SSEA-3; CD10; CD13; CD44; CD73; CD90; TNF-alpha receptor-1; toll like receptor 4; and/or the receptor for acetylated end products (RAGE). The fibroblasts may be cultured under hypoxia. When the fibroblasts are cultured under hypoxia they may secrete one or more factors selected from the group consisting of a) MCP-1; b) MIP1beta; c) IL-6; d) IL-8; e) GCP-2; f) HGF; g) KGF; h) FGF; i) HB-EGF; j) BDNF; k) TPO; l) RANTES; m) TIMP1; and n) a combination thereof.

Exosomes from fibroblasts may be administered instead of or in addition to the fibroblasts. The conditioned media and the fibroblasts may be administered concurrently or at separate times. The exosomes and the fibroblasts may be administered concurrently or at separate times.

In specific embodiments, the exosomes express one or more markers selected from the group consisting of CD63, CD9, MHC I, CD56, and a combination thereof.

Embodiments of the disclosure include isolated exosomes produced from fibroblasts cultured in vitro under hypoxic conditions. The exosomes may express one or more markers selected from the group consisting of CD63, CD9, MHC I, CD56, and a combination thereof. Any exosomes may be formulated as a pharmaceutical composition.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

In reviewing the detailed disclosure which follows, and the specification more generally, it should be borne in mind that all patents, patent applications, patent publications, technical publications, scientific publications, and other references referenced herein are hereby incorporated by reference in this application, in their entirety to the extent not inconsistent with the teachings herein. It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise.

### I. Examples of Definitions

In accordance with the presently disclosed invention, the word “comprising” is synonymous with “including,” “having,” “containing,” or “characterized by.” These terms are inclusive and open-ended and do not exclude additional, unrecited elements or method steps. In accordance with the presently disclosed invention, the phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole. In accordance with the presently disclosed invention, the phrase “consisting essentially of” limits the scope of a claim to the specified materials or steps, plus those that do not materially affect the basic and novel characteristic(s) of the claimed subject matter. It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

Some definitions are provided below to clarify concepts disclosed in the specification and to help one of skill in the art in the practice of the methods of the disclosure:

In accordance with the invention presented herein, the words “cell culture” and “culturing of cells” refer to the maintenance and propagation of cells and includes human, human-derived and animal cells in vitro.

In accordance with the disclosure presented herein, the term “cell culture medium” refers to the maintenance of cells in culture in vitro. For some cell types, the medium may also be sufficient to support the proliferation of the cells in culture. A medium according to the present disclosure provides nutrients such as energy sources, amino acids and inorganic ions. Additionally, it may contain a dye like phenol red, sodium pyruvate, several vitamins, free fatty acids, antibiotics, anti-oxidants and trace elements. For culturing the fibroblast cells that are possessive of anti-nociceptive activity according to the present disclosure, any standard medium such as Iscove's Modified Dulbecco's Media (IMDM), alpha-MEM, Dulbecco's Modified Eagle Media (DMEM), RPMI Media and McCoy's Medium may be used.

The term “acute pain” is defined as the pain caused by the injury of skin, body structure or internal organs and/or noxious stimulation of the diseases, or the pain caused by the abnormal function of muscle or internal organs that does not produce real tissue injury.

The term “chronic pain” is defined as the pain that lasts a period of time that exceeds the common course or healing time of acute diseases, or that is associated with the chronic pathological processes that cause continuous pain, or that relapses for several months or years with certain interval. If pain still exists after treatment that should cure the disease, such pain can be regarded as chronic pain. The time duration that the pain lasts depends on the nature of pain and the treatment process associated with pain. If the pain exceeds common treatment process, then this pain is chronic. Chronic pain includes but not limits to headache, facial pain, neck pain, shoulder pain, thoracic pain, abdominal pain, back pain, waist pain, lower limb pain, muscle and bone pain, somatoform disorder associated pain, visceral pain, painful diabetic neuropathy, vascular pain, gout, arthritis pain, cancer pain, autonomic nerve reflex pain, the pain caused by infectious diseases such as AIDS and herpes zoster, the pain caused by autoimmune disease such as rheumatism, the pain caused by acute or chronic inflammation, postoperative pain and post-burning pain. The intrathecal administration of fibroblasts disclosed by this invention can efficiently treat the chronic pain defined as above, and the drugs disclosed by this invention can be used to treat hyperalgia accompanied with other diseases, including hyperalgesia, allodynia, algesia enhancement and pain memory enhancement. This invention will improve the treatment of pain.

The term “neck pain, back pain, shoulder pain” represent the pain caused by acute or chronic muscle strain and bone joint degeneration and injury. The common diseases that cause neck, shoulder and upper limb pain include cervicoshoulder myofascitis, neck desmitis, cervical spondylopathy, scapulohumeral periarthritis, thoracic outlet syndrome, external humeral epicondylitis, etc. Alternatively, these terms represent the pain cause by autoimmune diseases rheumatoid arthritis, ankylosing spondylitis and rheumatic arthritis. Other diseases that can cause neck pain, back pain and shoulder pain are tumors on neck and shoulder, neuritis, arteriovenous disease and various infections as well as referred pain induced by lesions of thoracic and abdominal organs.

The term “thoracic, abdominal, and back pain” represent the pain caused by diseases in thoracic and abdominal organs and thoracic and abdominal wall tissues, including but not limiting to intercostal neuralgia, intercostal chondritis, angina pectoris, abdominal pain (acute abdominal organ pain) and waist and back myofascial pain syndrome.

The term “waist pain, lower limb pain” represent low back, lumbosacral, sacroiliac, hip, buttocks and lower limb pain. Generally, waist and lower limb pain is not independent disease, but the common feature of various diseases, with diverse clinical manifestation and complex etiology. Such pain is mainly induced by degeneration and injury, including but not limiting to the pain involving lumbar disc herniation, acute lumbar sprain, ischialgia, osteoporosis, third lumbar trans-verse process syndrome, piriformis syndrome, knee osteoarthritis, coccygodynia and calcanodynia.

The term “muscle and bone pain” includes but not limits to myofascial pain, trauma-caused pain and chronic regional pain syndrome.

The term “painful diabetes” represents the pain caused by nerve injury concurrent with diabetes. The nerve injury in diabetes is caused at least partly by blood flow reduction and hyperglycemia. Some diabetes patients do not suffer neuropathy, while others suffer this disease at early stage. Diabetic neuropathy can be divided into mononeuropathy that involves one or several lesion sites and systemic polyneuropathy. The polyneuropathy can be dispersive and symmetrical, generally and mainly involving mode of sensation (Merrit's Textbook of Neurology, the 9^{th} version). The manifestation of diabetic neuropathy includes plant nerve dysfunction, and cause dysregulation involving heart, smooth muscle and gland, resulting in hypotension, diarrhea, constipation and impotence. Diabetic neuropathy often develops in stages. The early stage takes place in nerve ending area. Plant neuropathy or sensory neuropathy occurs in feet and brain neuropathy occurs in face and periocular area with intermittent pain and the sense of tingling. In the following stages, the pain become more severe and occurs more frequently. Finally, when analgesia happens in one area, the disease develops into painless neuropathy. Due to lack of pain as the sign of injury, the risk of severe tissue damage is greatly increased.

The term “visceral pain” includes but not limits to the pain of inflammatory bowel syndrome (IBS), with or without chronic fatigue syndrome (CFS), inflammatory bowel disease (IBD) and interstitial cystitis.

The term “vascular pain” represents the pain generated by the following one or more factors. Firstly, improper perfusion of tissue, resulting in temporary or continuous ischemia, e.g the ischemia in limb muscles during physical exercise. Secondly, delayed change, e.g. ulcer or gangrene in skin or abdominal organs. Thirdly, the sudden and accelerated change of diameter of great vessels, e.g. the change of arterial aneurysm. Fourthly, aortic rupture, resulting in blood spillover and the stimulation of nociceptive fibers in peritoneum or pleura parietal layers. Fifthly, strong cramp caused by the severe stimulation of artery endothelium by intra-arterial injection. Sixthly, the damage of venous return, leading to a large number of edema of rapidly expanded fascia compartment (Bonica's Management of Pain, Volume 1 (the 2^{nd} version)). The examples include but not limit to arteriosclerosis obliterans, thromboangiitis angiitis, acute arterial closure, embolism, congenital arteriovenous aneurysm, vasospasm diseases, Rayaud's disease, acrocyanosis, acute venous closure, thrombophlebitis, varicosity and lymphedema.

The term “cancer pain” represents the pain occurs during the development process of malignant tumor. Currently, it is thought that there are three mechanisms of cancer pain, i.e. the pain caused directly by cancer development, the pain caused after cancer treatment and the concurrent painful diseases of cancer patients.

The term “autonomic nerve reflex pain” represents the pain caused by “reflex sympathetic dystrophy”. For reflex sympathetic dystrophy, after the body suffers acute or chronic injury, severe ambulatory pain occurs and the body is sensitive to the sense of touch and pain, probably accompanied with edema and blood disorder, following symptoms like skin and musculoskeletal nutrition dystrophia and atrophy.

The term “postoperative pain” represents a complex physiological response of body to the disease itself and the tissue injury caused by operation, showing an unpleasant psychological and behavior experience.

The term “arthritis pain” includes but not limits to the pain caused by osteoarthritis, rheumatoid arthritis, joint ankylosing spondylitis, psoriatic arthropathy, gout, pseudo gout, infectious arthritis, tendinitis, bursitis, bone damage and joint soft tissue inflammation.

The term “postherpetic neuralgia” represents the subcutaneously long-standing severe pain in rash site after the healing of the rash of herpes zoster.

The term “nociceptive pain” represents the pain caused by the tissue injury delivered by nociceptors, or the pain caused by the extended excitement of nociceptors. The pain caused by the extended excitement of nociceptors can be induced by both the persisting noxious stimulation of nociceptors and the sensitization thereof, or they can be induced by these factors and extended by their persistence, various reflex mechanisms and other factors.

The term “algesia” represents the neuromechanism for detecting noxious stimulation. Algesia involves two steps: the transduction of noxious stimulation by peripheral nerve ending and delivering these signals to central nervous system.

### II. General Embodiments

The disclosure provides means of treating pain through administration of fibroblasts and/or fibroblast derivatives at a concentration and frequency sufficient to reduce pain and/or delay its onset. In one embodiment of the disclosure, treatment of discogenic pain is provided by administration of an effective amount of fibroblasts and/or fibroblast derivatives into the intravertebral nucleus pulposus. In another embodiment, methods include administration of supernatant from a plurality of fibroblasts, and in one embodiment the supernatant comprises concentrated microvesicles that may be exosomes, for example.

For the practice of the methods of the disclosure, a particular embodiment includes the administration of fibroblast cells (such as intradiscally) and/or fibroblast derivatives at concentrations sufficient to treat an individual with a discogenic medical condition and having pain or at risk for pain. Without being bound to theory, administration of the fibroblasts may be in the form of the cells themselves, extracts of the cells, lysates, and/or nucleic acid compositions thereof; in specific embodiments the administration imparts the ability to reduce and/or reverse pain. The relief of the pain may or may not be total relief. A therapeutic activity may function through any means, although in specific embodiments it may be through restoration of antinociceptive neural pathways, inhibition of nociceptive signaling, reduction of inflammation, protection of neural cells from cellular death, stimulation of neural regeneration, and/or providing transfer of genetic material, for example.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In a particular embodiment, cells are cultured in the cell culture system that comprises a cell culture medium, such as in a culture vessel, in particular cases a cell culture medium supplemented with a substance suitable for culturing the cells in a manner so as to endow an ability to prevent, inhibit progression, delay the onset of, reduce the intensity of, and/or reverse Leigh Syndrome.

The disclosure provides means of reducing various types of pain by administration of fibroblasts and/or fibroblast derivatives. In a certain embodiment, the disclosure provides that fibroblasts, or one or more derivatives and/or one or more components thereof, possess an ability to inhibit pain, including discogenic pain, when administered in a manner allowing the fibroblasts or component(s) or derivative(s) thereof to access directly into the nucleus pulposus tissue (as an example). Other means of introducing fibroblasts, or derivatives thereof, for therapeutic utility according to the disclosure include intrathecal injections, intraventricular injections, topical, intravenous, subcutaneous, intra-arterial, intradermal, intra-rectal, and use of specialized delivery devices, such as an Ommya reservoir. The fibroblasts and/or derivatives thereof may be delivered locally or systemically.

Various definitions of pain in general are provided for the practice of the methods of the disclosure. In a broad definition, pain is an unpleasant feeling that is different from touch, pressure, heat and cold. Terms such as sharp pain, dull pain, aching pain, stabbing pain, cutting pain or burning pain are often used by individuals to describe pain, and the methods encompassed herein provide relief for any type of pain. Generally, the pain induced by nerve injury and showing hyperalgesia is referred as “neuropathic” pain, and the pain caused from the stimulating nociceptor is referred as “nociceptive” pain. For the practice of the current methods, administration of fibroblasts and/or derivatives is performed for both types of pain. In a particular embodiment, fibroblasts, or products thereof, such as supernatant, purified proteins from supernatants, microvesicles, or exosomes are administered for reducing pain, such as pain generating in the lumbar back area, for example.

Further classifications of pain are described in more detail. For example, clinically, pain can be divided into two classes: acute pain and chronic pain, and the methods of the disclosure may be used for either or both. Acute pain may be induced by the damage of skin, body structure or internal organs and/or noxious stimulation caused by diseases, or by the abnormal function of muscle or organ that does not generate actual tissue injury. Chronic pain may be defined as one kind of pain that lasts a period of time that exceeds the common course or healing time of acute diseases, or that is associated with the chronic pathological processes that cause continuous pain, or that relapses for several months or years with certain interval. If pain still exists after treatment that should cure the disease, such pain can be regarded as chronic pain, in specific embodiments. For the purpose of this disclosure, chronic pain can be chronic non-palliative or recurrent. The differences between the definition of acute pain and chronic pain may not only be semantic differences, but also may have clinic correlation. For example, if acute pain cannot be well-controlled, it can develop to chronic pain. Acute pain is different from chronic pain in the aspects of etiological mechanism, pathology and diagnoses and treatment. In contrast to the transiency of acute pain, chronic pain may be induced by the chronic pathological processes in body structure and internal organs, or by peripheral or central nervous system or the extended and sometimes permanent dysfunction thereof. In addition, chronic pain is sometimes attributed to psychological mechanisms and/or environmental factors. Generally, acute pain is non-neuropathic pain and includes common diseases such as arthritis pain, musculoskeletal pain, postoperative pain and fibromyalgia. The majority of such pain is thought to be caused by soft tissue and bone injury, for example arthritis pain, musculoskeletal pain and postoperative pain, and causes inflammatory reactions in normally functional nervous system, with pain just the consequence of inflammatory process. Chronic pain includes neuropathic pain, inflammatory pain and cancer pain, which is associated with hyperalgesia and/or allodynia, wherein hyperalgesia means elevated sensitivity to typical noxious stimulation while allodynia means elevated sensitivity to typical non-noxious stimulation. Somatogenic pain is caused by peripheral sensory nerve injury or infection, including but not limiting to pain caused by peripheral nerve injury, herpesvirus infection, diabetes, causalgia, blood vessel or plexus avulsion, neuralgia, amputation and nodular vasculitis. Neuropathic pain can also be induced by the nerve injury caused by chronic environmental poisoning, infection of human immunodeficiency virus, hypothyroidism, uremia or vitamin deficiency. Its clinical manifestation include but not limit to inflammatory pain, osteoarthritis pain, trigeminal neuralgia, cancer pain, diabetic neuropathy, restless legs syndrome, postherpetic neuralgia, causalgia, brachial vessel plexus avulsion, occipital neuralgia, gout, phantom limb, burn and other forms of neuralgia, neurological and spontaneous pain syndrome. Neuropathic pain is generally considered as chronic pain caused by peripheral or central nervous system injury or diseases. The medical conditions related to neuropathic pain include long-term peripheral or central neuron sensitization, central sensitization associated with nervous system inhibitory and/or exciting function injury as well as abnormal interaction between parasympathetic and sympathetic nervous system. Many clinical symptoms are related with neuropathic pain or form the basis of neuropathic pain, including for example diabetes, postoperative pain of amputation, lower back pain, cancer, chemical injury or toxin, other serious surgeries, peripheral nerve injury caused by traumatic injury compression, nutrition deficiency, infection such as herpes zoster and HIV. There are numerous causes of pain for which the methods of the disclosure are useful in addressing and one of skill in the art may utilize routine optimization to identify optimum dosages and treatment regiments. The disclosed methods may be used in the treatment of pain medical conditions, which include (but not limit to): headache, facial pain, neck pain, shoulder pain, back pain, thoracic pain, abdominal pain, dorsopathy, waist pain, lower limb pain, muscle and bone pain, body pain, vascular pain, gout, arthritis pain, somatoform disorder associated pain, visceral pain, the pain caused by infectious diseases such as AIDS and post-herpetic neuralgia, pain associated with multiple bone pain, sickle cell anemia, autoimmune disease, multiple sclerosis or inflammation acute or chronic inflammatory pain, cancer pain, neuropathic pain, injury or surgery caused pain, cancer pain, nociceptive pain, diabetes, peripheral neuropathies, post-herpetic neuralgia, trigeminal neuralgia, waist or cervix radiculopathy, glossopharyngeal neuralgia, autonomic nerve reflex pain, reflex sympathetic dystrophy, nerve root avulsion, cancer, chemical injury, toxin, nutrition deficiency, virus or bacteria infection, degenerative osteoarthropathy or the combination thereof.

Although the disclosure provides a general embodiment that fibroblasts possess anti-nociceptive activities for the varieties of pains mentioned above, in one embodiment intradiscally-administered fibroblasts may be used for the purpose of reduction and/or amelioration of back pain or any other type of pain. Conditions often causing pains in low back (that is, the lumbar) and pelvic area of a person are as follows, and methods for examination, diagnosis and/or treatment of these examples of diseases are well known in the art and are presented to allow the practitioner of the disclosure to perform the teachings of the disclosure.

Herniated Intervertebral Disc (HIVD)—Lumbar Radiculopathy. This condition is often called sciatica and accompanies neurotic disturbance (or disorder) in association with lower limbs to a certain extent. This condition may occur by stimulating the fifth lumbar nerve and the first sacral nerve due to extrusion of nucleus pulposus and, optionally, caused by direct nerve root compression and/or chemical stimulation of substances in the nucleus pulposus. Prevalence of HIVD is about 2% of total population and 10 to 25% among them with HIVD show continued symptoms of six weeks or longer. Cases requiring surgery may range from 5 to 10% of total disc patients. This condition generally expresses serious (acute) symptoms and is often accompanied with low back pain. Some patients say sometimes that previously suffered pain disappeared after having leg pain. The pain becomes increased during sitting, coughing or sneezing. It may be difficult to posture for convenience while reducing pain. When taking a pose bending knees and putting the same to the chest, the disc is expanded and the patient should bend as much as possible to increase an inner radius of a spinal cavity, thus helping easy the pain. There are typically pains from the hip toward posterior or postero-lateral parts up to the ankle or foot. For a neuromuscular disease at the center of lumbar (L1-L3), referred pain is expressed at anterior thigh but, usually, not extended below the knee. HIVD on this site is not higher than 5% of overall HIVD. Radiculopathy refers to disease of the spinal nerve roots (from the Latin radix for root). Radiculopathy produces pain, numbness, or weakness radiating from the spine. Radiculopathies are categorized according to which part of the spinal cord is affected. Thus, there are cervical (neck), thoracic (middle back), and lumbar (lower back) radiculopathies. Lumbar radiculopathy is also known a sciatica. Radiculopathies may be further categorized by what vertebrae they are associated with. For example, radiculopathy of the nerve roots at the level of the seventh cervical vertebra is termed C7 radiculopathy; at the level of the fifth cervical vertebra, C5 radiculopathy; at the level of the first thoracic vertebra, T1 radiculopathy. Spinal Stenosis. This refers to a condition wherein, owing to certain reasons, a spinal canal, nerve root canal or intervertebral cavity is narrowed to induce low back pain or cause a variety of complicated neuroses on legs. In general, the nucleus pulposus and fibrous ring begin to be degenerated after 30 years old, and thus, a part of intervertebral disc adhered to the spin is detached to remain bone spur. Simultaneously, posterior joint protrusion, vertebral arch, ligament flavum, may also be deformed and thickened to make all area around the spinal canal to be narrowed. Further, the spin is bent in front and rear directions to directly press the spinal cord and nerve roots and cause blood flow disorder, resulting in occurrence of symptoms. Low back pain is very often expressed, dislike lumbar intervertebral herniation, sensory disorder and weakened muscular strength as well as sharp, squeezing or burning pain arises around the hip and anus accompanied with sensory disorder and weakened muscular strength. Such symptoms as described above are generally worse under cold weather or during exercising but improved under warm weather or when a patient is at ease. For symptoms often arising and becoming serious, these symptoms disappear when the patient bends at the waist or stops walking but crouches down to rest, while being repeated by walking again. Such a condition as described above typically refers to as neurogenic intermittent claudication. As an extent of stenosis is increased, a walking distance may be shortened. Conditions of a sensory disorder, such as sensibility loss or numbness, may be expressed over a wide area of the body along with calf, ankle, knee, thigh, hip and inguinal regions. Anus dysfunction is one symptom. Degenerative Disc Disease: This refers to a disc condition with degraded mechanical and/or chemical properties of the intervertebral disc due to various causes including, i.e., ageing, trauma, high impacting activity, type of works, smoking, genetic factors, and so forth. A mechanical disorder having low back pain during bending or stretching the body and neurotic disorders having leg pain during sitting down or walking are expressed. A degenerative intervertebral disc disease often arises without specific symptoms. Patients with typical degenerative disc explain that a pain arises when getting up in the morning, however, disappears during walking about 1 hour. At a molecular level, disc degeneration associated with the aging process is generally associated with the loss of proteoglycan from the nucleus pulposus of the spinal discs and a reduction of the disc's ability to absorb shock between vertebrae. Although some affected patients may not exhibit symptoms, many affected patients suffer from chronic back or neck pain in addition to arm and/or leg pain. Pain associated with disc degeneration may become debilitating and may greatly reduce a patient's quality of life. In some embodiments, the degenerative disc disease can include an intervertebral disc herniation. As used herein “intervertebral disc herniation” includes local displacement of disc material beyond the limits of the intervertebral disc space. The disc material may be nucleus pulposus, cartilage, fragmented apophysical bone, annular tissue or any combination thereof. Displacement of disc material may put pressure on the exiting spinal nerve and/or cause an inflammatory reaction leading to radiculopathy, weakness, numbness, and/or tingling in the arms or legs. In general, most degenerative disc disease takes place in the lumbar area of the spine. Lumbar disc herniation occurs 15 times more often than cervical disc herniation, and it is one of the most common causes of lower back pain. The cervical discs are affected 8% of the time and the upper-to-mid-back (thoracic) discs only 1-2% of the time. Sometimes degenerative disc disease can lead to compression of the nerve roots of the spine resulting in very painful neurological symptoms. Nerve roots (large nerves that branch out from the spinal cord) may become compressed resulting in neurological symptoms, such as sensory or motor changes. For example herniation of the nucleus pulposus often is accompanied by lower back pain that worsens in the sitting position and pain that radiates to the lower extremities. The radiating pain, for example, in sciatica is often described as dull, burning or sharp pain, accompanied by intermittent sharp electric shock sensation, numbness, and tingling, motor or sensory defects of the respective nerve root and/or reflex abnormalities.

Spobdylolisthesis: In this condition the vertebra comprises multiple small bonds stacked in a tower form. Joint protrusions in a ring type ring placed at a rear part of the vertebra fix upper and lower bones. Spobdylolisthesis refers to the wherein the upper spinal bone slides and is forced out toward the front due to various causes such as damage of joint protrusions. Major causes may include degeneration of discs and joints, congenital spinal abnormality, accident, impact-derived fracture of spinal joint protrusion, and the like. In a case of standing up after sitting down or stretching (or bending) backward at the waist, low back pain is caused. When getting up in the morning, low back pain is caused. In a case of taking a stand for a long time or walking a long distance, it causes pains in the waist, hip and/or below knees. By passing a hand over the spine during straightening the waist body and touching the body, depressed parts are found. Walking with faltering steps like a duck is found. Facet Joint Syndrome: In this condition pain is generated through nerves distributed over a facet joint since a joint membrane of the facet joint sensitive to pain has acute trauma or degenerative modification, thus causing fracture of the face joint membrane or arthritis. Pain of which the position is not certainly detected or traced. The patient possesses symptom of strain from the hip to the posterior thigh (similar to intervertebral disc disorder). Radiating pain of the lower limbs is not usually broadened below the knees. Pain increasing when getting up in the morning, however, decreasing during activity. Pain decreasing during bending frontward, however, increasing during stretching and bending in lateral sides. Intervertebral disc herniation includes a rupture of the annulus fibrosis, through which the inner disc material (nucleus pulposus) extrudes, protrudes, bulges, migrates and/or re-herniates. Sometimes disc extrusions may be displaced so much that it has lost continuity with the parent disc. When this happens the extrusion is called sequestration. Thus, the methods of the present application can be used to treat pain associated with ruptures, protrusions, bulges, extrusions, re-herniation, and migration, fragmented, and/or sequestrated nucleus pulposus.

Embodiments of the disclosure include methods of reducing pain in an individual by administering to the individual effective amounts of fibroblasts and/or fibroblast derivatives systemically and/or locally. Although the pain may be of any kind, in specific embodiments the pain is selected from the group consisting of: a) neuropathic pain; b) nociceptive pain; c) phantom pain; d) psychogenic pain; e) incident pain; f) breakthrough pain; g) discogenic pain; h) idiopathic pain; and i) a combination thereof.

Fibroblasts and/or fibroblast derivatives utilized in methods herein may be of any kind but in specific embodiments they are selected from the group of tissues consisting of: a) bone marrow; b) perivascular tissue; c) adipose tissue; d) placental tissue; e) amniotic membrane; f) omentum; g) dental pulp; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; w) subepithelial umbilical cord tissue; x) foreskin tissue; and y) a combination thereof.

The fibroblasts (and by extension the derivatives therefrom in at least some cases) may have one or more particular characteristics. The cells may or may not express one or more markers, such as CXCR-4; CD-271; FGF-1 receptor; SSEA-3; CD10; CD13; CD44; CD73; CD90; TNF-alpha receptor-1; toll like receptor 4; and/or the receptor for acetylated end products (RAGE). In specific cases the fibroblasts are capable of producing HGF-1. The fibroblasts may possess ability to inhibit TNF-alpha production from an activated macrophage. The fibroblasts may express a higher amount of T cell proliferation inhibitory activity as compared to bone marrow derived mesenchymal stem cells when cultured under similar or identical conditions. The fibroblasts may express, relative to bone marrow-derived mesenchymal stem cells, increased levels of M-CSF under identical culture conditions. The fibroblasts may have the potential to differentiate into cells of a chondrogenic phenotype when cultured under chondrogenic inductive media. In specific cases, the fibroblasts are isolated from adipose tissue substantially free of blood that is capable of self-renewal and expansion in culture. The fibroblasts may have the potential to differentiate into cells of other phenotypes besides fibroblasts. In cases when fibroblasts are cultured under hypoxic conditions, the fibroblasts may secrete factors selected from the group consisting of: a) MCP-1; b) MIP1beta; c) IL-6; d) IL-8; e) GCP-2; f) HGF; g) KGF; h) FGF; i) HB-EGF; j) BDNF; k) TPO; 1) RANTES; m) TIMP1; and n) a combination thereof.

In particular embodiments, before administration the fibroblasts are passaged in culture, and in specific embodiments the fibroblasts maintain a normal karyotype upon passaging for more than about 15, 16, 17, 18, 19, 20, 21, 22, 23, 24, 25, or more cell doublings.

When the fibroblasts and/or fibroblast derivatives are administered to an individual, they may be administered by any suitable route but in specific embodiments they are administered intradiscally. The fibroblasts and/or fibroblast derivatives may be administered alone and/or conditioned media from culture of the fibroblasts may be administered. For example, exosomes from fibroblasts may be administered instead of fibroblasts or in addition to fibroblasts and/or fibroblast derivatives. The conditioned media and the fibroblasts and/or fibroblast derivatives may or may not be administered concurrently. Fibroblast derived exosomes and the fibroblasts and/or fibroblast derivatives may or may not be administered concurrently.

### III. Production of Fibroblast Derivatives

Embodiments of the disclosure include one or more compositions (including injectable compositions) for the treatment of discogenic pain, and in specific embodiments the compositions comprise exosomes from cultured fibroblasts. In specific embodiments the exosomes are generated by a process comprising the steps of: a) obtaining one or more fibroblast cells; b) culturing said fibroblast cells under conditions to allow for production of exosomes into culture media within which said fibroblast cell is cultured; c) extracting exosomes from the culture media. In particular embodiments the exosomes are administered to an individual in need of treatment. The fibroblasts from which the exosomes are derived may be derived from a biopsy, wherein a donor providing the biopsy is either the individual to be treated (autologous) or the donor is different from the individual to be treated (allogeneic). In specific cases the fibroblasts are cultured in a media allowing for fibroblast proliferation, and the media allowing for fibroblast proliferation may comprise one or more factors known to be mitogenic for fibroblasts, such as one or more factors selected from the group consisting of of: a) FGF-1; b) FGF-2; c) FGF-5; d) EGF; e) CNTF; f) KGF-1; g) PDGF; h) platelet rich plasma; i) TGF-alpha; j) HGF-1; and k) a combination thereof. In any case, fibroblasts may be cultured under hypoxia.

Exosomes may or may not be obtained from fibroblasts while the fibroblasts are in a proliferating state. In specific cases, exosomes are obtained from fibroblasts while the fibroblasts are cultured in a media comprising no proliferative factors or largely reduced levels of proliferation inducing growth factors and the growth factors may be undefined growth factors such as fetal calf serum, neonatal serum, cord blood serum, or platelet lysate, or the growth factors may be defined mitogens such as EGF, FGF-1, FGF-2, FGF-5. In specific embodiments, exosomes are collected from fibroblasts that have been cultured in 2-8% oxygen for at least 1 day. The amount of oxygen may be 2, 3, 4, 5, 6, 7, or 8% in the culture. A range of oxygen levels in culture may be 2-8, 2-7, 2-6, 2-5, 2-4, 2-3, 3-8, 3-7, 3-6, 3-5, 3-4, 4-8, 4-7, 4-6, 4-5, 5-8, 5-7, 5-6, 6-8, 6-7, or 7-8%. In specific embodiments, the cells are cultured for 1-15 days or 5-10 days. The cells may be cultured for 1, 2, 3, 4, 5, 6, 7, 8, 9, 10, 11, 12, 13, 14, 15, 16, 17, 18, 19, or 20 or more days. The cells may be cultured for a range of days that is 1-15, 1-14, 1-13, 1-12, 1-11, 1-10, 1-9, 1-8, 1-7, 1-6, 1-5, 1-4, 1-3, 1-2, 2-15, 2-14, 2-13, 2-12, 2-11, 2-10, 2-9, 2-8, 2-7, 2-6, 2-5, 2-4, 2-3, 3-15, 3-14, 3-13, 3-12, 3-11, 3-10, 3-9, 3-8, 3-7, 3-6, 3-5, 3-4, 4-15, 4-14, 4-13, 4-12, 4-11, 4-10, 4-9, 4-8, 4-7, 4-6, 4-5, 5-15, 5-14, 5-13, 5-12, 5-11, 5-10, 5-9, 5-8, 5-7, 5-6, 6-15, 6-14, 6-13, 6-12, 6-11, 6-10, 6-9, 6-8, 6-7, 7-15, 7-14, 7-13, 7-12, 7-11, 7-10, 7-9, 7-8, 8-15, 8-14, 8-13, 8-12, 8-11, 8-10, 8-9, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, 13-15, 13-14, or 14-15 days. In culture, the cells may be passaged for at least 1, 2, 3, 4, 5, 6, 7, 8, 9, 10, or more passages. In specific cases the fibroblasts are cultured in media selected from the group consisting of: a) Roswell Park Memorial Institute (RPMI-1640); b) Dublecco's Modified Essential Media (DMEM), c) Eagle's Modified Essential Media (EMEM), d) Optimem, and e) Iscove's Media.

In cases wherein exosomes from fibroblasts are utilized, the exosomes may be in a preparation, such as a preparation that comprises less than 5% polyethylene glycol. The exosomes may be purified using polyethylene glycol and/or using ultrafiltration. In some cases, polyethylene glycol is added to the exosomes after purification. The exosomes may or may not express one or more certain markers, such as markers selected from the group consisting of: a) CD63; b) CD9; c) MHC I; d) CD56; and e) a combination thereof.

In some cases, in addition or as an alternative to administering fibroblasts and/or exosomes from fibroblasts, an individual may be administered a sufficient amount of fibroblast lysate. Generation of lysate from cells is routine in the art.

In one embodiment, fibroblasts are cultured using means known in the art for preserving viability and proliferative ability of fibroblasts. The disclosed methods may be applied both for individualized autologous exosome preparations and for exosome preparations obtained from established cell lines, for experimental or biological use. In one embodiment, methods of the disclosure encompass the use of chromatography separation methods for preparing membrane vesicles, particularly to separate the membrane vesicles from potential biological contaminants, wherein the microvesicles are exosomes, and cells utilized for generating said exosomes are fibroblast cells. The exosomes are obtained and may be prepared for administration to one or more individuals in need thereof.

Indeed, the applicant has now demonstrated that membrane vesicles, particularly exosomes, could be purified, and possess ability to inhibit pain. In one embodiment, a strong or weak, preferably strong, anion exchange may be performed. In addition, in a specific embodiment, the chromatography is performed under pressure. Thus, more specifically, it may consist of high performance liquid chromatography (HPLC). Different types of supports may be used to perform the anion exchange chromatography. More preferably, these may include cellulose, poly(styrene-divinylbenzene), agarose, dextran, acrylamide, silica, ethylene glycol-methacrylate co-polymer, or mixtures thereof, e.g., agarose-dextran mixtures. To illustrate this, it is possible to mention the different chromatography equipment composed of supports as mentioned above, particularly the following gels: SOURCE POROS® SEPHAROSE®, SEPHADEX®, TRISACRYL®, TSK-GEL SW OR PW®, SUPERDEX® TOYOPEARL HW and SEPHACRYL®, for example, which are suitable for the application of this invention. Therefore, in a specific embodiment, this invention relates to a method of preparing membrane vesicles, particularly exosomes, from a biological sample such as a tissue culture containing fibroblasts, comprising at least one step during which the biological sample is treated by anion exchange chromatography on a support selected from cellulose, poly(styrene-divinylbenzene), silica, acrylamide, agarose, dextran, ethylene glycol-methacrylate co-polymer, alone or in mixtures, optionally functionalized.

In addition, to improve the chromatographic resolution, within the scope of the disclosure, one can use supports in bead form. In particular embodiments, these beads have a homogeneous and calibrated diameter, with a sufficiently high porosity to enable the penetration of the objects under chromatography (i.e. the exosomes). In this way, given the diameter of exosomes (generally between 50 and 100 nm), to apply the invention, one can use high porosity gels, particularly between 10 nm and 5 μm, such as between approximately 20 nm and approximately 2 μm, including between about 100 nm and about 1 μm. For the anion exchange chromatography, the support used may be functionalized using a group capable of interacting with an anionic molecule. Generally, this group comprises an amine that may be ternary or quaternary, which defines a weak or strong anion exchanger, respectively. Within the scope of this disclosure, one can utilize a strong anion exchanger. In this way, according to the disclosure, a chromatography support as described above, functionalized with quaternary amines, may be used. Therefore, according to a more specific embodiment of the disclosure, the anion exchange chromatography is performed on a support functionalized with a quaternary amine. In specific cases, this support is selected from poly(styrene-divinylbenzene), acrylamide, agarose, dextran and silica, alone or in mixtures, and may be functionalized with a quaternary amine. Examples of supports functionalized with a quaternary amine include the gels SOURCEQ. MONO Q, Q SEPHAROSE®, POROS® HQ and POROS® QE, FRACTOGEL® TMAE type gels and TOYOPEARL SUPER® Q gels.

One example of a support to perform the anion exchange chromatography comprises poly(styrene-divinylbenzene). An example of this type of gel that may be used within the scope of this disclosure is SOURCE Q gel, particularly SOURCE 15 Q (Pharmacia). This support offers the advantage of very large internal pores, thus offering low resistance to the circulation of liquid through the gel, while enabling rapid diffusion of the exosomes to the functional groups, which are particularly important parameters for exosomes given their size. The biological compounds retained on the column may be eluted in different ways, particularly using the passage of a saline solution gradient of increasing concentration, e.g. from 0 to 2 M. A sodium chloride solution may particularly be used, in concentrations varying from 0 to 2 M, for example. The different fractions purified in this way may be detected by measuring their optical density (OD) at the column outlet using a continuous spectro-photometric reading. As an indication, under the conditions used in the examples, the fractions comprising the membrane vesicles were eluted at an ionic strength comprised between approximately 350 and 700 mM, depending on the type of vesicles.

Different types of columns may be used to perform this chromatographic step, according to requirements and the volumes to be treated. For example, depending on the preparations, it is possible to use a column from approximately 100 μl up to 10 ml or greater. In this way, the supports available have a capacity which may reach 25 mg of proteins/ml, for example. For this reason, a 100 μl column has a capacity of approximately 2.5 mg of proteins which, given the samples in question, allows the treatment of culture supernatants of approximately 2 l (which, after concentration by a factor of 10 to 20, for example, represent volumes of 100 to 200 ml per preparation). It is understood that higher volumes may also be treated, by increasing the volume of the column, for example. In addition, to perform this invention, it is also possible to combine the anion exchange chromatography step with a gel permeation chromatography step. In this way, according to a specific embodiment of the disclosure, a gel permeation chromatography step is added to the anion exchange step, either before or after the anion exchange chromatography step. In this embodiment, the permeation chromatography step takes place after the anion exchange step. In addition, in a specific variant, the anion exchange chromatography step is replaced by the gel permeation chromatography step. The present application demonstrates that membrane vesicles may also be purified using gel permeation liquid chromatography, particularly when this step is combined with an anion exchange chromatography or other treatment steps of the biological sample, as described in detail below.

To perform the gel permeation chromatography step, a support selected from silica, acrylamide, agarose, dextran, ethylene glycol-methacrylate co-polymer or mixtures thereof, e.g., agarose-dextran mixtures, may be used. As an illustration, for gel permeation chromatography, a support such as SUPERDEX® 200HR (Pharmacia), TSK G6000 (TosoHaas) or SEPHACRYL® S (Pharmacia) may be used. The process according to the disclosure may be applied to different biological samples. In particular, these may comprise a biological fluid from a subject (bone marrow, peripheral blood, etc.), a culture supernatant, a cell lysate, a pre-purified solution or any other composition comprising membrane vesicles.

In this respect, in a specific embodiment of the disclosure, the biological sample is a culture supernatant of membrane vesicle-producing fibroblast cells.

In addition, according to one embodiment of the disclosure, the biological sample is treated, prior to the chromatography step, to be enriched with membrane vesicles (enrichment stage). In this way, in a specific embodiment, this disclosure relates to a method of preparing membrane vesicles from a biological sample, characterized in that it comprises at least: a) an enrichment step, to prepare a sample enriched with membrane vesicles, and b) a step during which the sample is treated by anion exchange chromatography and/or gel permeation chromatography.

In one embodiment, the biological sample is a culture supernatant treated so as to be enriched with membrane vesicles. In particular, the biological sample may be comprised of a pre-purified solution obtained from a culture supernatant of a population of membrane vesicle-producing cells or from a biological fluid, by treatments such as centrifugation, clarification, ultrafiltration, nanofiltration and/or affinity chromatography, particularly with clarification and/or ultrafiltration and/or affinity chromatography. Therefore, one method of preparing membrane vesicles according to this disclosure more particularly comprises the following steps: a) culturing a population of membrane vesicle (e.g. exosome) producing cells under conditions enabling the release of vesicles, b) a step of enrichment of the sample in membrane vesicles, and c) an anion exchange chromatography and/or gel permeation chromatography treatment of the sample.

As indicated above, the sample (e.g. supernatant) enrichment step may comprise one or more centrifugation, clarification, ultrafiltration, nanofiltration and/or affinity chromatography steps on the supernatant. In a first specific embodiment, the enrichment step comprises (i) the elimination of cells and/or cell debris (clarification), possibly followed by (ii) a concentration and/or affinity chromatography step. In one specific embodiment, the enrichment step comprises an affinity chromatography step, optionally preceded by a step of elimination of cells and/or cell debris (clarification). An example of an enrichment step according to this disclosure comprises (i) the elimination of cells and/or cell debris (clarification), (ii) a concentration and (iii) an affinity chromatography. The cells and/or cell debris may be eliminated by centrifugation of the sample, for example, at a low speed, preferably below 1000 g, between 100 and 700 g, for example. Preferred centrifugation conditions during this step are approximately 300 g or 600 g for a period between 1 and 15 minutes, for example.

The cells and/or cell debris may also be eliminated by filtration of the sample, possibly combined with the centrifugation described above. The filtration may particularly be performed with successive filtrations using filters with a decreasing porosity. For this purpose, filters with a porosity above 0.2 μm, e.g. between 0.2 and 10 μm, may be used. It is particularly possible to use a succession of filters with a porosity of 10 μm, 1 μm, 0.5 μm followed by 0.22 μm.

A concentration step may also be performed, such as in order to reduce the volumes of sample to be treated during the chromatography stages. In this way, the concentration may be obtained by centrifugation of the sample at high speeds, e.g., between 10,000 and 100,000 g, to cause the sedimentation of the membrane vesicles. This may comprise a series of differential centrifugations, with the last centrifugation performed at approximately 70,000 g. The membrane vesicles in the pellet obtained may be taken up with a smaller volume and in a suitable buffer for the subsequent steps of the process. The concentration step may also be performed by ultrafiltration. In fact, this ultrafiltration allows both to concentrate the supernatant and perform an initial purification of the vesicles. According to a particular embodiment, the biological sample (e.g., the supernatant) is subjected to an ultrafiltration, preferably a tangential ultrafiltration. Tangential ultrafiltration comprises concentrating and fractionating a solution between two compartments (filtrate and retentate), separated by membranes of determined cut-off thresholds. The separation is carried out by applying a flow in the retentate compartment and a transmembrane pressure between this compartment and the filtrate compartment. Different systems may be used to perform the ultrafiltration, such as spiral membranes (Millipore, Amicon), flat membranes or hollow fibres (Amicon, Millipore, Sartorius, Pall, GF, Sepracor). Within the scope of the invention, the use of membranes with a cut-off threshold below 1000 kDa, preferably between 300 kDa and 1000 kDa, or even more preferably between 300 kDa and 500 kDa, is advantageous.

The affinity chromatography step can be performed in various ways, using different chromatographic support and material. It is advantageously a non-specific affinity chromatography, aimed at retaining (i.e., binding) certain contaminants present within the solution, without retaining the objects of interest (i.e., the exosomes). It is therefore a negative selection. In some cases, an affinity chromatography on a dye is used, allowing the elimination (i.e., the retention) of contaminants such as proteins and enzymes, for instance albumin, kinases, dehydrogenases, clotting factors, interferons, lipoproteins, or also co-factors, etc. More preferably, the support used for this chromatography step is a support as used for the ion exchange chromatography, functionalized with a dye. As specific example, the dye may be selected from Blue SEPHAROSE® (Pharmacia), YELLOW 86, GREEN 5 and BROWN 10 (Sigma). The support may be agarose. It should be understood that any other support and/or dye or reactive group allowing the retention (binding) of contaminants from the treated biological sample can be used in the instant disclosure.

In one embodiment a membrane vesicle preparation process within the scope of this disclosure comprises the following steps: a) the culture of a population of membrane vesicle (e.g. exosome) producing cells under conditions enabling the release of vesicles, b) the treatment of the culture supernatant with at least one ultrafiltration or affinity chromatography step, to produce a biological sample enriched with membrane vesicles (e.g. with exosomes), and c) an anion exchange chromatography and/or gel permeation chromatography treatment of the biological sample. In a certain embodiment, step b) above comprises a filtration of the culture supernatant, followed by an ultrafiltration, such as tangential. In another embodiment, step b) above comprises a clarification of the culture supernatant, followed by an affinity chromatography on dye, such as on Blue SEPHAROSE®.

In addition, after step c), the material harvested may, if applicable, be subjected to one or more additional treatment and/or filtration stages d), particularly for sterilization purposes. For this filtration treatment stage, filters with a diameter less than or equal to 0.3 μm may be used, or for example, less than or equal to 0.25 μm. Such filters have a diameter of 0.22 μm, for example.

After step d), the material obtained is, for example, distributed into suitable devices such as bottles, tubes, bags, syringes, etc., in a suitable storage medium. The purified vesicles obtained in this way may be stored cold, frozen or used extemporaneously. Therefore, a specific preparation process within the scope of the disclosure comprises at least the following steps: c) an anion exchange chromatography and/or gel permeation chromatography treatment of the biological sample, and d) a filtration step, particularly sterilizing filtration, of the material harvested after stage c). In a first variant, the process according to the disclosure comprises: c) an anion exchange chromatography treatment of the biological sample, and d) a filtration step, particularly sterilizing filtration, on the material harvested after step c). In some cases, instead of being stored the material may be used for one or more individuals in the absence of a prior storage step.

In another variant, the process according to the disclosure comprises: c) a gel permeation chromatography treatment of the biological sample, and d) a filtration step, particularly sterilizing filtration, on the material harvested after step c). According to a third variant, the process according to the disclosure comprises: c) an anionic exchange treatment of the biological sample followed or preceded by gel permeation chromatography, and d) a filtration step, particularly sterilizing filtration, on the material harvested after step c).

Further embodiments include a method of optimizing one or more pain inhibiting therapeutic factors production from fibroblast cultures through the use of filters that separate compositions based on electrical charge, size and/or ability to elute from an adsorbent. Numerous techniques are known in the art for purification of therapeutic factors and concentration of agents. For some particular uses fibroblast derived compounds are sufficient for use as culture supernatants of the cells in media. Currently media useful for this purpose include Roswell Park Memorial Institute (RPMI-1640), Dublecco's Modified Essential Media (DMEM), Eagle's Modified Essential Media (EMEM), Optimem, and Iscove's Media.

In one embodiment, therapeutic factors capable of inhibiting discogenic pain, or other types of pain, are derived from tissue culture that may comprise exosomes, or may not comprise exosomes but comprise one or more factors capable of stimulating an antinociceptive effect. In such an embodiment, culture conditioned media may be concentrated by filtering/desalting means known in the art including use of Amicon filters with specific molecular weight cut-offs, said cut-offs may select for molecular weights higher than 1 kDa to 50 kDa. Supernatant may alternatively be concentrated using means known in the art such as solid phase extraction using C18 cartridges (Mini-Spe-ed C18-14%, S.P.E. Limited, Concord ON). The cartridges may be prepared by washing with methanol followed by deionized-distilled water. Up to 100 ml of fibroblast conditioned media supernatant may be passed through each of these specific cartridges before elution, and it is understood of one of skill in the art that larger cartridges may be used. After washing the cartridges, material adsorbed is eluted, such as with 3 ml methanol, evaporated under a stream of nitrogen, re-dissolved in a small volume of methanol, and stored at 4° C. Before testing the eluate for activity in vitro, the methanol is evaporated under nitrogen and replaced by culture medium. The C18 cartridges may be used to adsorb small hydrophobic molecules from the fibroblast conditioned supernatant and allows for the elimination of salts and other polar contaminants. It may, however be desired to use other adsorption means in order to purify certain compounds from the supernatant. The concentrated supernatant may be assessed directly for biological activities useful for the practice of this disclosure, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5×54 cm glass column and equilibrated with 3 column volumes of the same buffer. Fibroblast cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

### IV. Administration of Fibroblasts and/or Fibroblast Derivatives

In some embodiments of the disclosure, pain (including back pain) is treated by administration of sufficient amounts of fibroblast cells and/or derivatives thereof, such as via intraventricular injection. In one embodiment, intraventricular injection is accomplished via an Ommaya reservoir. Use of Ommaya reservoir for administration of stem cells and other cellular therapies has been previously reported and is incorporated by reference [13-18].

In one embodiment of the disclosure, fibroblasts and/or fibroblast derivatives are administered in proximity to a pain marker. While generally the disclosure encompasses intradiscal administration of fibroblasts for cessation of pain, in some embodiments, specific localization of the fibrobasts is desired in proximity to originating source of pain. Pain marker(s) are considered contributors in various ways to (or otherwise indicative of) the generation or transmission of discogenic pain. One such pain marker relates to the presence of nociceptors. Normally, intervertebral discs are substantially avascular and only sparsely innervated at the outer margins of the disc annulus. These unmyelinated, substance P (SP) or calcitonin gene-related peptide (CGRP) comprising fibers are typically unresponsive and termed silent nociceptors. SP and CGRP are believed to be the sensory transmitters of nociceptive information. As degeneration proceeds, nerves can follow microvessels and grow deeper into discs, which may occur for example either peripherally or via the endplate. This nerve and vessel in-growth is facilitated by degeneration-related decreases in disc pressure and proteoglycan content. Thus, by administering a diagnostic agent that binds SP or CGRP, the SP and CGRP conjugate will have an abnormally high concentration and, therefore, imaging will show a high signal to noise ratio (e.g., 2/1, 3/1, 4/1, 5/1, 6/1, 7/1, 8/1, 9/1, 10/1 etc.) and the pain generator will be clearly imaged and diagnosed in the area of “hot spots”. This provides a means of localizing administration of fibroblasts. In some embodiments, fibroblasts are temporarily immobilized by administration using a biodegradable matrix.

In some embodiments, fibroblasts and/or fibroblast derivatives may be administered at locations that are identified using agents capable of localizing origination of pain in the vertebral column. Diagnostic agents may be labeled in vivo by administering a label that binds with the pain marker. In some embodiments, the neuronal pain marker that can be labeled includes, without limitation: TRK-alpha; anti-TRK .alpha. antibody; nerve growth factor (NGF); anti-NGF antibody; NGF antagonist; anti-NGF antagonist antibody; PGP 9.5; SYN; peripherin; or other form of nerve antibodies or related materials in general. Other materials such as neurofilament 200 kDa (NF200) may also be the target of such labeling and subsequent imaging. In some embodiments, endogenous substances such as TrkA or NGF may be targeted as the pain markers for labeling, or related antibodies or other substances having particular binding affinity or specificity to such substances may be bound to them in the area of pain and then thereafter provide the binding site for targeted labels to be subsequently delivered. In this regard, it is to be appreciated that various forms of binding agents are broadly contemplated hereunder this description. Nerves usually accompany blood vessels, but can be found as isolated nerves fibers in the disc matrix. These non-vessel-associated fibers found in back pain patients have been observed to express growth-associated protein 43 (GAP43) as well as SP. Small disc neurons contain CGRP and also express the high-affinity nerve growth factor (NGF) receptor, tyrosine kinase A (trkA). Disc inflammation has been observed to cause an increase in CGRP positive neurons. A recent study showed that NGF is expressed in microvascular blood vessels in a painful lumbar disc, and that there are trkA (TRK-alpha) expressing nerve fibers adjacent to the vessels that enter painful discs primarily through the endplate. Along with nerves growing into degenerated discs are specialized nerve support cells termed ‘glia’ or Schwann cells localized using glial fibrillary acidic protein (GFAP). Accordingly, various such materials may provide the requisite binding affinity or specificity to painful regions (or highly innervated regions) to play the role as the pain marker for the diagnostic agent to bind to or they may be labeled and then administered to identify the pain marker or the pain generator. In one embodiment, for example, TrkA antibody (or other binding agent) is labeled and delivered for binding and visualization to the pain generator. In another embodiment, NGF itself is labeled and delivered as a diagnostic agent, which binds the pain marker TrkA. Some of these agents are further described in Published PCT Patent Applications are herein incorporated in their entirety by reference thereto: WO 2004/032870; WO 2004/058184; WO 2004/073653; WO 2004/096122; and WO 2005/000194.

### V. Obtaining Fibroblasts and Manipulations Thereof

The fibroblasts and/or fibroblast derivatives utilized in the disclosure may be generated, in one embodiment, by outgrowth from a biopsy of the recipient's own skin (in the case of autologous preparations), or skin of healthy donors (for allogeneic preparations). In some embodiments fibroblasts are used from young donors, and in any event the fibroblasts may come from infants, children, adolescents, and/or adults. In one embodiment, fibroblasts are transfected with genes to allow for enhanced growth and overcoming of the Hayflick limit. This may or may not occur subsequent to derivation of cells and expansion in culture using standard cell culture techniques.

The fibroblasts and/or fibroblast derivatives may be obtained by any suitable manner. As one example, skin tissue (dermis and epidermis layers) may be biopsied from a subject's post-auricular area. In one embodiment, the starting material is comprised of three 3-mm punch skin biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. In one embodiment, after arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6 (Helidelburg, Germany). After digestion, Initiation Growth Media (IMDM, GA, 10% Fetal Bovine Serum (FBS)) is added to neutralize the enzyme, cells are pelleted by centrifugation and re-suspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask. Cells are incubated at 37±2.0° C. with 5.0±1.0% CO₂ and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask. Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies. Cells are incubated at 37±2.0° C. with 5.0±1.0% CO₂ and passaged every three to five days in the T-500 flask and every five to seven days in the ten layer cell stack (10 CS). Cells should not remain in the T-500 flask for more than 10 days prior to passaging. Quality Control (QC) release testing for safety of the Bulk Drug Substance includes sterility and endotoxin testing. When cell confluence in the T-500 flask is .gtoreq.95%, cells are passaged to a 10 CS culture vessel. Alternately, two Five Layer Cell Stacks (5 CS) or a 10 Layer Cell Factory (10 CF) can be used in place of the 10 CS. 10 CS. Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO₂ and fed with fresh Complete Growth Media every five to seven days. Cells should not remain in the 10 CS for more than 20 days prior to passaging. In one embodiment, the passaged dermal fibroblasts are rendered substantially free of immunogenic proteins present in the culture medium by incubating the expanded fibroblasts for a period of time in protein free medium, Primary Harvest When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

In some embodiments, when large numbers of cells are required after receiving cell count results from the primary 10 CS harvest, an additional passage into multiple cell stacks (up to four 10 CS) is performed. For additional passaging, cells from the primary harvest are added to a 2 L media bottle containing fresh Complete Growth Media. Resuspended cells are added to multiple cell stacks and incubated at 37±2.0.degree. C. with 5.0±1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above. The method decreases or eliminates immunogenic proteins be avoiding their introduction from animal-sourced reagents. To reduce process residuals, cells are cryopreserved in protein-free freeze media, then thawed and washed prior to prepping the final injection to further reduce remaining residuals. If additional Drug Substance is needed after the harvest and cryopreservation of cells from additional passaging is complete, aliquots of frozen Drug Substance—Cryovial are thawed and used to seed 5 CS or 10 CS culture vessels. Alternatively, a four layer cell factory (4 CF), two 4 CF, or two 5 CS can be used in place of a 5 CS or 10 CS. A frozen cryovial(s) of cells is thawed, washed, added to a 2 L media bottle containing fresh Complete Growth Media and cultured, harvested and cryopreserved as described above. The cell suspension is added Cell confluence must be 80% or more prior to cell harvest. At the completion of culture expansion, the cells are harvested and washed, then formulated to contain 1.0-2.7×10⁷ cells/mL, with a target of 2.2×10⁷ cells/mL. Alternatively, the target can be adjusted within the formulation range to accommodate different indication doses. The drug substance consists of a population of viable, autologous human fibroblast cells suspended in a cryopreservation medium consisting of Iscove's Modified Dulbecco's Medium (IMDM) and Profreeze-CDM.™. (Lonza, Walkerville, Md.) plus 7.5% dimethyl sulfoxide (DMSO). Alternatively, a lower DMSO concentration may be used in place of 7.5% or CryoStor.™. CS5 or CryoStor.™. CS10 (BioLife Solutions, Bothell, Wash.) may be used in place of IMDM/Profreeze/DMSO. In addition to cell count and viability, purity/identity of the Drug Substance is performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD104, integrin .beta.4 chain, is a 205 kDa transmembrane glycoprotein which associates with integrin .alpha.6 chain (CD49f) to form the .alpha.6/.beta.4 complex. This complex has been shown to act as a molecular marker for keratinocyte cells (Adams and Watt 1991).

Cell count and viability may be determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead. The method was custom developed specifically for use in determining purity/identity of autologous cultured fibroblasts. Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage dependent cells in suspension culture. They are designed to produce large cell yields in small volumes. In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37±2.0° C. with 5.0±1.0% CO₂ incubator to allow for adherence of cells to the carriers. After the attachment period, the speed of the spin plate is increased (30-120 RPM). Cells are fed with fresh Complete Growth Media every one to five days, or when media appears spent by color change. Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsinizationtrypsiziation and reseeding. Alternatively, if enough cells are produced from the initial T-175 or T-500 flask, the cells can be directly seeded into the scale-up amount of microcarriers. After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore.™. (GE Healthcare, Piscataway, N.J.) or coated/uncoated polystyrene, such as 2D MicroHex.™. (Nunc, Weisbaden, Germany), Cytodex® (GE Healthcare, Piscataway, N.J.) or Hy-Q Sphere.™. (Thermo Scientific Hyclone, Logan, Utah).

In another embodiment, cells can be processed on poly blend 2D microcarriers such as BioNOC II® and FibraCel® using an automatic bellow system, such as FibraStage.™. (New Brunswick Scientific, Edison, N.J.) or BelloCell.®. (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above. Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsinization for harvest upon completion of the cell expansion stage.

Alternatively, the ACE system can be a scaled down, single lot unit version comprised of a disposable component that consists of cell growth surface, delivery tubing, media and reagents, and a permanent base that houses mechanics and computer processing capabilities for heating/cooling, media transfer and execution of the automated programming cycle. Upon receipt, each sterile irradiated ACE disposable unit will be unwrapped from its packaging and loaded with media and reagents by hanging pre-filled bags and connecting the bags to the existing tubing via aseptic connectors. The process continues as follows: a) Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place; b) After approximately three days, the cells within the pre-growth chamber are trypsinized and introduced into the cell tower itself, which is pre-filled with Complete Growth Media. Here, the “bubbling action” caused by CO₂ injection force the media to circulate at such a rate that the cells spiral downward and settle on the surface of the discs in an evenly distributed manner; c) For approximately seven days, the cells are allowed to multiply. At this time, confluence will be checked (method unknown at time of writing) to verify that culture is growing. Also at this time, the Complete Growth Media will be replaced with fresh Complete Growth Media. CGM will be replaced every seven days for three to four weeks. At the end of the culture period, the confluence is checked once more to verify that there is sufficient growth to possibly yield the desired quantity of cells for the intended treatment; d) If the culture is sufficiently confluent, it is harvested. The spent media (supernatant) is drained from the vessel. PBS will then is pumped into the vessel (to wash the media, FBS from the cells) and drained almost immediately. Trypsin-EDTA is pumped into the vessel to detach the cells from the growth surface. The trypsin/cell mixture is drained from the vessel and enter the spin separator. Cryopreservative is pumped into the vessel to rinse any residual cells from the surface of the discs, and be sent to the spin separator as well. The spin separator collects the cells and then evenly re-suspend the cells in the shipping/injection medium. From the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

In another embodiment, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option.

In one embodiment, fibroblasts are pre-activated by contact with one or more growth factor-comprising mixtures, and the mixture or composition comprises growth factors selected from the group consisting of transforming growth factors (TGF), fibroblast growth factors (FGF), platelet-derived growth factors (PDGF), epidermal growth factors (EGF), vascular endothelial growth factors (VEGF), insulin-like growth factors (IGF), platelet-derived endothelial growth factors (PDEGF), platelet-derived angiogenesis factors (PDAF), platelet factors 4 (PF-4), hepatocyte growth factors (HGF) and mixtures thereof. More preferably, the growth factors are transforming growth factors (TGF), platelet-derived growth factors (PDGF) fibroblast growth factors (FGF) and mixtures thereof. In specific cases, the growth factors are selected from the group consisting of transforming growth factors beta (TGF-beta), platelet-derived growth factors BB (PDGF-BB), basic fibroblast growth factors (bFGF) and mixtures thereof. In another embodiment of the disclosure, the growth factor comprising composition(s) are injected simultaneously with, or subsequent to, injection of fibroblasts. The fibroblasts may be autologous, allogeneic, or xenogeneic.

In some embodiments, a platelet plasma composition is administered together with the fibroblasts and/or fibroblast derivatives and/or is administered subsequent to administration of the fibroblasts and/or fibroblast derivatives. In specific cases, the composition comprises, consists essentially of, or consists of platelets and plasma and may be derived from bone marrow or peripheral blood or a combination thereof. Methods of the present disclosure may use platelet plasma compositions from either or both of these sources, and either platelet plasma composition may be used to regenerate a nucleus and/or annulus in need thereof in addition to providing pain relief. Further, the platelet plasma composition may be used with or without concentrated bone marrow (BMAC). By way of example, when inserted into the annulus, 0.05-2.0 cc of platelet plasma composition may be used, and when inserted into the nucleus, 0.05-3.0 cc of the platelet plasma composition may be used. Platelets are non-nucleated blood cells that as noted above are found in bone marrow and peripheral blood. They have several important functions such as controlling bleeding and tissue healing. Useful factors from platelets that promote tissue healing include growth factors that they produce, such as platelet-derived growth factor (PDGF), transforming growth factor beta (TGF-beta), fibroblast growth factor (FGF), insulin-like growth factor-1 (IGF-1), connective tissue growth factor (CTGF) and vascular endothelial growth factor (VEGF). Many of these platelet proteins and molecules are cytokines and are important for cell signaling and immunomodulation.

In various embodiments of the present disclosure, the platelet plasma composition may be obtained by sequestering platelets from whole blood or bone marrow through centrifugation into three strata: (1) platelet rich plasma; (2) platelet poor plasma; and (3) fibrinogen. When using platelets from one of the strata, e.g., the platelet rich plasma (PRP) from blood, one may use the platelets whole or their contents may be extracted and concentrated into a platelet lysate through a cell membrane lysis procedure using thrombin and/or calcium chloride, for example. When choosing whether to use the platelets whole or as a lysate, one may consider the rate at which one desires regeneration and/or tissue healing (which may include the formation of scar tissue without regeneration or healing of a herniated or torn disc). In some embodiments the lysate will act more rapidly than the PRP (or platelet poor plasma from bone marrow). Notably, platelet poor plasma that is derived from bone marrow has a greater platelet concentration than platelet rich plasma from blood, also known as platelet poor/rich plasma, (“PP/RP” or “PPP”). PP/RP or PPP may be used to refer to platelet poor plasma derived from bone marrow, and in some embodiments, preferably PP/RP is used or PRP is used as part of the composition for disc regeneration. (By convention, the abbreviation PRP refers only to compositions derived from peripheral blood and PPP (or PP/RP) refers to compositions derived from bone marrow.) In various embodiments, the platelet plasma composition, which may or may not be in the form of a lysate, may serve one or more of the following functions: (1) to release/provide growth factors and cytokines for tissue regeneration; (2) to reduce inflammation; (3) to attract/mobilize cell signaling; (4) to initiate fibroblast repair of damaged annulus through fibroblast growth factors (FGF); (5) to stabilize disc annulus; (6) to repair annulus disc tears; (7) to stimulate revascularization to a disc; and (8) to stimulate stem cell activation. Additionally, by combining platelet therapy with stem cells, there can be synergy with respect to reducing back pain.

### VI. Kits of the Disclosure

Any of the cellular and/or non-cellular compositions described herein or similar thereto may be comprised in a kit. In a non-limiting example, one or more reagents for use in methods for preparing cellular therapy may be comprised in a kit. Such reagents may include cells; media; and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### REFERENCES

1. Rubin, D. I., Epidemiology and risk factors for spine pain. Neurol Clin, 2007. 25(2): p. 353-71.

2. Luo, X., et al., Estimates and patterns of direct health care expenditures among individuals with back pain in the United States. Spine (Phila Pa 1976), 2004. 29(1): p. 79-86.

3. Pengel, L. H., et al., Acute low back pain: systematic review of its prognosis. BMJ, 2003. 327(7410): p. 323.

4. Smith, C. and K. Grimmer-Somers, The treatment effect of exercise programmes for chronic low back pain. J Eval Clin Pract, 2010. 16(3): p. 484-91.

5. Woolf, A. D. and B. Pfleger, Burden of major musculoskeletal conditions. Bull World Health Organ, 2003. 81(9): p. 646-56.

6. Koes, B. W., M. W. van Tulder, and S. Thomas, Diagnosis and treatment of low back pain. BMJ, 2006. 332(7555): p. 1430-4.

7. Mehling, W. E., et al., Clinical decision rule for primary care patient with acute low back pain at risk of developing chronic pain. Spine J, 2015. 15(7): p. 1577-86.

8. Chou, R., et al., Diagnosis and treatment of low back pain: a joint clinical practice guideline from the American College of Physicians and the American Pain Society. Ann Intern Med, 2007. 147(7): p. 478-91.

9. van Tulder, M., et al., Chapter 3. European guidelines for the management of acute nonspecific low back pain in primary care. Eur Spine J, 2006. 15 Suppl 2: p. S169-91.

10. Roelofs, P. D., et al., Non-steroidal anti-inflammatory drugs for low back pain. Cochrane Database Syst Rev, 2008(1): p. CD000396.

11. Naesdal, J. and K. Brown, NSAID-associated adverse effects and acid control aids to prevent them: a review of current treatment options. Drug Saf, 2006. 29(2): p. 119-32.

12. Herrmann, W. A. and M. S. Geertsen, Efficacy and safety of lornoxicam compared with placebo and diclofenac in acute sciatica/lumbo-sciatica: an analysis from a randomised, double-blind, multicentre, parallel-group study. Int J Clin Pract, 2009. 63(11): p. 1613-21.

13. Baek, W., et al., Stem cell transplantation into the intraventricular space via an Ommaya reservoir in a patient with amyotrophic lateral sclerosis. J Neurosurg Sci, 2012. 56(3): p. 261-3.

14. Clemons-Miller, A. R., et al., Intrathecal cytotoxic T-cell immunotherapy for metastatic leptomeningeal melanoma. Clin Cancer Res, 2001. 7(3 Suppl): p. 917s-924s.

15. Quattrocchi, K. B., et al., Pilot study of local autologous tumor infiltrating lymphocytes for the treatment of recurrent malignant gliomas. J Neurooncol, 1999. 45(2): p. 141-57.

16. Merchant, R. E., et al., Intralesional infusion of lymphokine-activated killer (LAK) cells and recombinant interleukin-2 (rIL-2) for the treatment of patients with malignant brain tumor. Neurosurgery, 1988. 23(6): p. 725-32.

17. Naganuma, H., et al., Complete remission of recurrent glioblastoma multiforme following local infusions of lymphokine activated killer cells. Case report. Acta Neurochir (Wien), 1989. 99(3-4): p. 157-60.

18. Yoshida, S., et al., Local administration of autologous lymphokine-activated killer cells and recombinant interleukin 2 to patients with malignant brain tumors. Cancer Res, 1988. 48(17): p. 5011-6.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of treating or preventing pain in an individual, comprising the step of administering to the individual an effective amount of fibroblasts and/or derivatives thereof and/or conditioned media from culture of said fibroblasts.

2. The method of claim 1, wherein the administration is local or systemic to the individual.

3. The method of claim 1 or 2, wherein the administration is to the spine of the individual.

4. The method of claim 1, 2, or 3, wherein the administration is intradiscally in the individual.

5. The method of any one of claims 1-4, wherein the pain is acute or chronic.

6. The method of any one of claims 1-5, wherein the individual is receiving an additional treatment.

7. The method of claim 6, wherein the additional treatment is for pain.

8. The method of any one of claims 1-7, wherein the pain is selected from the group consisting of a) neuropathic pain; b) nociceptive pain; c) phantom pain; d) psychogenic pain; e) incident pain; f) breakthrough pain; g) discogenic pain; h) idiopathic pain; and i) a combination thereof.

9. The method of any one of claims 1-8, wherein the fibroblast derivative comprises lysate and/or exosomes.

10. The method of claim 9, wherein the exosomes are obtained following culture of the fibroblasts under suitable conditions.

11. The method of any one of claims 1-10, wherein the fibroblasts express CXCR-4; CD-271; FGF-1 receptor; SSEA-3; CD10; CD13; CD44; CD73; CD90; TNF-alpha receptor-1; toll like receptor 4; and/or the receptor for acetylated end products (RAGE).

12. The method of any one of claims 1-11, wherein the fibroblasts are cultured under hypoxia.

13. The method of claim 12, wherein when the fibroblasts are cultured under hypoxia they secrete one or more factors selected from the group consisting of a) MCP-I; b) MIP1beta; c) IL-6; d) IL-8; e) GCP-2; f) HGF; g) KGF; h) FGF; i) HB-EGF; j) BDNF; k) TPO; l) RANTES; m) TIMP1; and n) a combination thereof.

14. The method of any one of claims 1-13, wherein exosomes are administered instead of the fibroblasts.

15. The method of any one of claims 1-14, wherein the conditioned media and the fibroblasts are administered concurrently or at separate times.

16. The method of any one of claims 1-15, wherein the exosomes and the fibroblasts are administered concurrently or at separate times.

17. The method of any one of claims 1-16, wherein the exosomes express one or more markers selected from the group consisting of CD63, CD9, MHC I, CD56, and a combination thereof.

18. Isolated exosomes produced from fibroblasts cultured in vitro under hypoxic conditions.

19. The exosomes of claim 18, wherein the exosomes express one or more markers selected from the group consisting of CD63, CD9, MHC I, CD56, and a combination thereof.

20. The exosomes of claim 18 or 19, said exosomes formulated as a pharmaceutical composition.
